(19) United States
(12) Patent Application Publication
(10) Pub. No.: US 2004/0018636 A1
(43) Pub. Date: Jan. 29, 2004

(54) Immunological assay test device

(76) Inventors: David F. Zhou, San Diego, CA (US); Nai Shu (Sue) Wang, San Diego, CA (US); Claudia J.R. Shen, San Diego, CA (US); Angela J.Q. Shen, San Diego, CA (US)

Correspondence Address: CHARMASSON & BUCHACA, 1545 HOTEL CIRCLE SOUTH, SUITE 150, SAN DIEGO, CA, 92108-3412, US

(21) Appl. No.: 10/206,512

(22) Filed: Jul. 25, 2002

(51) Int. Cl. G01N 33/558

(52) U.S. Cl. 436/514

(57) ABSTRACT

A device using polychromatic assay strips in the detection of drug residue in urine and in connection with other such immunoassay tests mounts two sets of parallel strips back-to-back. Each set of strips is visible through a window on the front or back faces of the device. The specimen fluid is deposited through an aperture in the device enclosure and carried to the test strips by a wick in the form of a piece of blotting paper. One of the test strips visible through a separate window is used to detect adulteration of the specimen.

### FIELD OF THE INVENTION

This invention relates to analytical test devices for analyzing body fluids using immunochromotography and more particularly to apparatuses for concurrent performance of multiple analysis on a common test fluid by contacting the fluid to discrete polychromatic test strips.

### BACKGROUND OF THE INVENTION

The prior art offers several business card-sized devices for preliminary testing of the presence of drugs in urine samples as typically disclosed in U.S. Pat. No. 6,403,383 Casterlin et al., which patent is incorporated in this specification by this reference.

A recent Supreme Court decision that upheld the constitutionality of drug testing by public schools and other similar institutions has greatly increased demands for quick and efficient devices for the preliminary detection of traces of drugs in urine samples. Accordingly, there is an urgent need for an improved test device that can be used to perform a larger number of drug tests on a small amount of fluid specimen and requires a relatively small level of operator's skill.

### SUMMARY OF THE INVENTION

The principal and secondary objects of this invention are to provide and immunochromotographic test device that can be used to perform a dozen or more drug-detecting tests on a small volume of urine or other test fluid including the detection of adulterated specimens.

These and other valuable objects are achieved by a device mounting two sets of parallel test strips in a back-to-back configuration. Each set of strips is visible through windows on the front and back faces of the device. A small amount of specimen fluid can be deposited through and aperture in the device enclosure. A sheet of blotting paper of other high capillarity material delivers an aliquot of the fluid to a number of reagent test strips. One of the strips visible through a separate window is used to detect adulteration of the specimen. A small amount of fluid specimen can be deposited over the aperture by means of a pipette or the bottom portion of the device can be dipped in a specimen fluid container.

### BRIEF DESCRIPTION OF THE DRAWING

FIG. 1 is a front elevational view of the immunological assay device according to the invention;

FIG. 2 is a back elevational view thereof;

FIG. 3 is a side expanded view of the components of the device; and

FIG. 4 is a cross-sectional view of the lower portion taken along line 4-4 of FIG. 1.

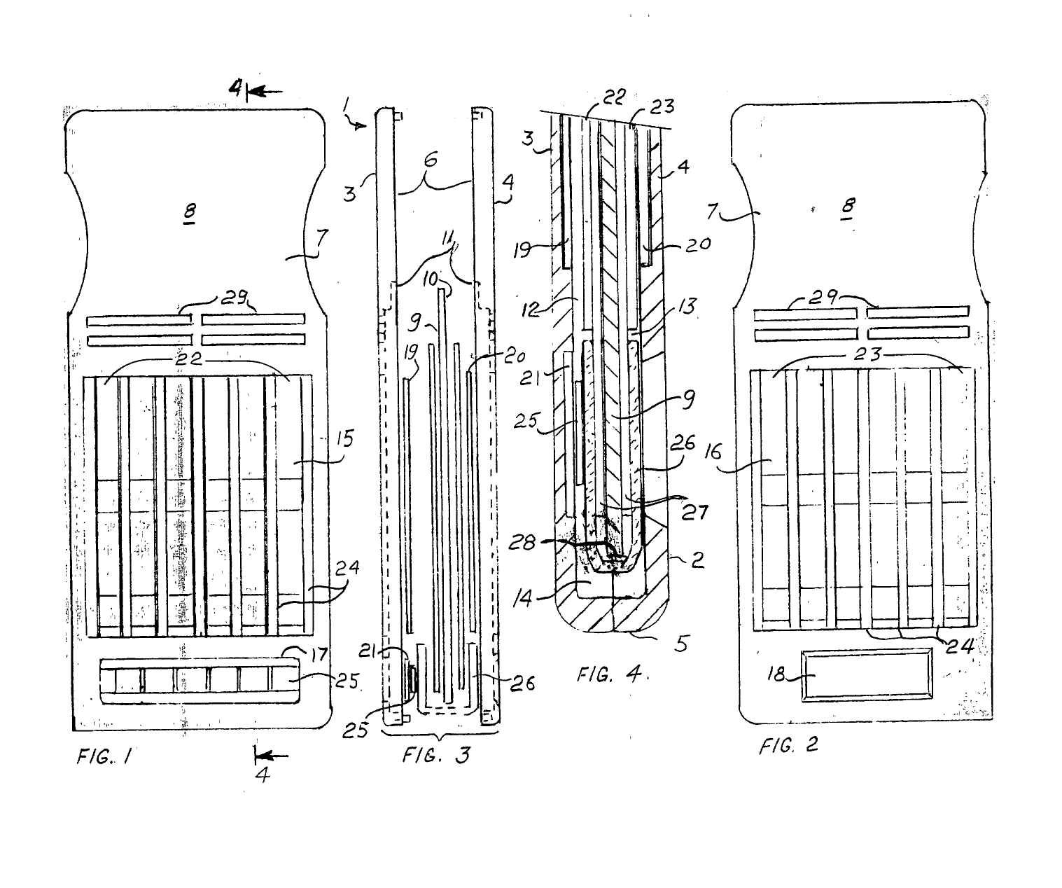

### DESCRIPTION OF THE PREFERRED EMBODIMENT OF THE INVENTION

Referring now to the drawing, there is shown an immunochromotographic device 1 packaged in an enclosure 2 having the approximate size of a business card. The enclosure comprises a planar front wall 3, a planar back wall 4 symmetrical with the front wall, and a relatively narrow peripheral wall 5 half of which is formed integrally with the front wall and the other half with the back wall so as to form two symmetrical clam shells 6. The upper third section of the device forms a convenient handle and provides a writing surface 8 to carry a date, a specimen identification or other pertinent information.

A planar septum 9 is positioned in the middle of the enclosure, having its upper edge 10 pinched between recessed areas 11 of the two clam shells 6. The septum forms a first chamber 12 in combination with the front wall 3 and a second chamber 13 in combination with the back wall 4. The septum 9 is shaped and dimensioned to leave a gap or opening 14 between the two chambers at the bottom of the enclosure. Two large symmetrical windows 15, 16 are cut through the front and back walls respectively. Two smaller, horizontally oblong windows 17, 18 are located in the lower parts of the front and back walls. The two large windows 15, 16 and the smaller window 17 in the front wall are sealed by transparent panes 19, 20, 21. The small window 18 in the back wall remains unsealed. A set of six chromotographic reagent test strips 22, 23 is mounted against each front and back face of the septum 9 where they are held and cradled in a parallel spaced-apart configuration by series of ribs 24. A special strip 17 specifically formulated to detect adulteration of a fluid specimen is positioned orthogonally to the other strips behind the special smaller window 17. A wick 26 preferably made of a piece of blotting paper or other high-capillarity material passes through the opening between the two chambers, extends over the entire unsealed window 18 in the back wall and is in contact with the lower portions 27 of the vertical test strips as well as the special strip 25 as the wick is folded over the lower edge 28 of the septum. Sets of ventilation slots 29 are provided in the front and back walls in the upper section of the two chambers 12, 13.

Accordingly, aliquots of a small volume of fluid specimen dropped into the sample well consitituted by the back window 18 are delivered to the various test strips. The polychromatic indication of the analytic results are visible through the two large windows 15, 16 and the special window 17.

It should be noted that the back window 18 could be eliminated, and the front special window 17 unsealed to act as a sample well so long as the special strip 25 does not completely seal the aperture of that special window. It should also be noted that instead of dropping a small volume of fluid specimen through the window/sample well, the lower part of the device could be dipped into the fluid specimen container as was taught by the prior art.

While the preferred embodiment of the invention has been described, modifications can be made and other embodiments may be devised without departing from the spirit of the invention and the scope of the appended claims.

What is claimed is:

1. A device for simultaneously performing a plurality of immunochromatographic tests on a fluid specimen, said device comprising:
a flat enclosure having a front wall, a back wall and a narrow peripheral wall;
each of said front and back walls having at least one windown cut therethrough, one of said windows being unsealed;
a medium septum defining a first chamber in association with the front wall and a second chamber in association with the back wall, said septum being shaped and dimensioned to leave an opening between said first and second chambers;
a wick passing through said opening and extending into said first and second chambers; and
in each of said chambers, a plurality of chromatographic test strips, each having a portion in contact with said wick and a color-indicating portion visible through one of said windows;
whereby a small amount of said fluid specimen passing through said unsealed window can seep through said wick to contact each of said strips.

2. The device of claim 1, wherein a distinct one of said strips is formulated to detect adulteration of said fluid specimen.

3. The device of claim 2, wherein, in one of said chambers, a number of said strips are laid side-by-side in a parallel, spaced-apart arrangement; and
said distinct strips lies perpendicularly to said number of strips.

4. The device of claim 3, wherein a special one of said windows is shaped and dimensioned to expose only one of said strips.

5. The device of claim 3, wherein a special one of said windows is shaped and dimensioned to expose only said distinct strip.

6. The device of claim 4, wherein said unsealed window is positioned on a face of said device opposite said special window.

7. The device of claim 1, wherein said wick consists essentially of a sheet of blotting paper.

8. The device of claim 6, wherein said sheet is folded over one edge of said septum.

9. The device of claim 1, wherein a plurality of said windows are sealed by transparent panes.

10. The device of claim 3, wherein said septum comprises parallel and spaced-apart ribs cradling said strips.
